(19) United States
(12) Patent Application Publication
(10) Pub. No.: US 2001/0051616 A1
(43) Pub. Date: Dec. 13, 2001

(54) METHOD OF LESSENING THE RISK OF VERTEBRAL FRACTURES

(76) Inventors: DAVID B. KARPF, EDISON, NJ (US); THOMAS P. CAPIZZI, NORTH BRUNSWICK, NJ (US); HUI QUAN, EDISON, NJ (US); ARTHUR C. SANTORA II, WATCHUNG, NJ (US); ASHLEY J. YATES, WESTFIELD, NJ (US)

Correspondence Address: JOANNE M GIESSER, PATENT DEPARTMENT, MERCK AND COMPANY INC, P O BOX 2000, RAHWAY, NJ, 070650907

(21) Appl. No.: 08/389,860

(22) Filed: Feb. 17, 1995

(51) Int. Cl. A61K 31/66; A61K 31/44

(52) U.S. Cl. 514/108

(57) ABSTRACT

Alendronate, a bisphosphonate, when administered daily over a substantial period of time, can reduce the rate of vertebral fractures in post-menopausal women. Further it can reduce the number and severity of fractures. Also, administration of alendronate can prevent spinal deformity, and loss in height.

### SUMMARY OF THE INVENTION

This invention is related to a method of lessening the risk of vertebral fractures in post-menopausal women by administering an effective amount of alendronate, a bisphosphonate.

### BACKGROUND OF THE INVENTION

Osteoporosis is a metabolic disease characterized by an age-related decrease in bone mass and strength. The condition primarily affects post-menopausal women, although it may affect elderly men as well. The most common clinical manifestations of osteoporosis are fractures of the vertebrae, hip, and wrist.

Osteoporosis-related fractures are very common, occurring in some 27% of women over the age of 65 and some 60% of those over 80 years of age. Vertebral fractures often go undiagnosed, although they are frequently accompanied by pain, and may limit the patient's ability to perform daily activities. Multiple vertebral fractures may lead to a kyphotic posture, chronic back pain and disability.

A number of therapies are currently used for the prevention and treatment of osteoporosis, including hormone replacement (estrogen), calcitonin, etidronate (a bisphosphonate), ipriflavone, fluoride, Vitamin D, and calcium. The extent of treatment varies worldwide.

While it has been reported that some of the aforementioned treatment agents can increase bone mineral density (BMD), there is no established correlation between increased BMD and a decrease in vertebral fractures. While low BMD is correlated with an increased rate of fracture, a higher BMD is not necessarily correlated with an decrease in fracture. For example, fluoride has been shown to increase BMD, but the rate of hip fracture also increases.

### DESCRIPTION OF THE INVENTION

It has been found in accordance with this invention that the administration of alendronate (4-amino-1-hydroxy-butylidene- 1,1-bisphosphonate) is usefull in lessening the risk of vertebral fractures in osteoporotic post-menopausal women. Thus, this invention provides a method of reducing the risk of vertebral fractures by administering an effective amount of alendronate or a pharmaceutically acceptable salt to osteoporotic women. Furthermore, this risk reduction is maintained and even lowered with the long-term administration of alendronate. Another object of this invention is to reduce the risk of spinal deformity by administering an effective amount of alendronate or pharmaceutically acceptable salt thereof for a substantial period of time. Another aspect of this invention is to prevent the loss of height by administering an effective amount of alendronate or a pharmaceutically acceptable salt thereof for a substantial period of time. Yet another aspect of this invention is a method of reducing the severity of vertebral fractures in patients who sustain such a fracture by administering alendronate for a substantial period of time prior to sustaining the fracture.

It has been surprisingly found that the incidence of vertebral fractures can be reduced when an effective amount of alendronate is administered over a substantial period of time. The decrease in the risk of vertebral fractures is estimated to be at least about 40%, preferably at least about 45%, and even more preferably at least about 48%; this decrease was found to be statistically significant when compared to placebo. When the total number of vertebral fractures (as opposed to the number of patients with fractures) was calculated, alendronate produces at least about 50%, preferably at least about 60% and even more preferably at least about 63% reduction in vertebral fracture rate per 100 patients when compared to placebo. Likewise, alendronate produces a statistically significant decrease in the progression of vertebral deformity as compared to placebo patients. Furthermore, the risk rate for vertebral fractures (compared to placebo) is less after three years administration than after one or two years administration. Also, in women who received alendronate but also sustained a vertebral fracture, the severity of the fractures were less than those sustained by women receiving placebo.

It has also been found in accordance with this invention that the increase in bone mineral density observed with the administration of alendronate is positively associated with a decrease in vertebral fractures, a decrease in spinal deformity and a retention of height. This indicates that when administered for a substantial period of time, alendronate not only decreases bone resorption, but also acts positively to produce a strengthened bone.

The woman who receives alendronate according to this invention is suffering from osteoporosis, i.e., has a bone mineral density (BMD) which is at least about two or two and one-half standard deviations below the norm of premenopausal women.

### DESCRIPTION OF THE FIGURE

FIG. 1 is a graph showing the time response profile for decrease in stature of all patients in placebo and alendronate groups. The mean change and SE are noted.

FIG. 2 is a graph showing the time response profile for decrease in stature in patients having an incident vertebral fracture during the study. The mean change and SE are shown.

Throughout the specification and claims the following definitions shall apply:

“Effective amount” shall mean at least the amount of alendronate required to provide a decrease in the risk of fracture, but less that a toxic amount.

“Substantial period of time” means an amount of time which is long enough to allow the bones of the patient to have an increased bone mineral density (BMD) and strength such that they are more resistant to fractures. A typical substantial period of time is a long period of time, and is in excess of two years, and preferably in excess of three years.

“Substantially daily” means that the administration is intended to be daily, but the patient may occasionally inadvertently skip doses, such that the overall effect is not different from that observed when a patient receives the dosage daily.

“Elderly” means that age is equal to or greater than 65 years.

“Non-elderly” means that age is less than 65 years.

“PRY” means person-years-at risk, and is calculated by multiplying the number of patients in a trial by the number of years of the trial.

Alendronate may be prepared according to any of the processes described in U.S. Pat. Nos. 5,019,651, 4,992,007, and U.S. application Ser. No. 08/286,151, filed Aug. 4, 1994, each of which is hereby incorporated by reference. The pharmaceutically acceptable salts of alendronate include salts of alkali metals (e.g., Na, K). aikali earth metals (e.g., Ca), salts of inorganic acids, such as HCl and salts of organic acids such as citric acid and amino acids. Sodium salt forms are preferred, particularly the monosodium salt trihydrate formn.

The compounds of the present invention can be administered in oral dosage forms such as tablets, capsules (each of which includes sustained release or timed release formulations), pills, powders, granules, elixirs, paste, tinctures, suspensions, syrups, dissolved forms and emulsions. Likewise they may be administered in an intravenous (bolus or infusion), intraperitoneal, subcutaneous, or intramuscular form, all using forms well known to those of ordinary skill in the pharmaceutical arts. An effective but non-toxic amount of the compound desired can be used as a fracture-preventing agent.

Patients preferably will receive alendronate substantially daily for a substantial period of time in order for the effect to be observable. This means that the patient will receive alendronate at least one-half of the days in a treatment period, with the treatment period lasting at least one year, and is preferably longer, up to and exceeding two, three or more years. In a preferred embodiment, the patient will receive alendronate substantially daily for at least three years in order to experience the greatest benefit. It is envisioned that a patient receiving such a long-term therapy may experience occasional periods when alendronate is not administered; but since alendronate has a long active life in the bone, this is considered within the scope of the invention provided that the patient receives alendronate at least one-half of the days in the preceding six month period. Also, it is within the scope of this invention that the alendronate be administered on a cyclical regime, i.e., the patient may receive alendronate for a given period of time (for example, one day, weekly, monthly, semi-monthly, or for several months) then may be taken off the alendronate (and may or may not be given additional bone-promoting or bone absorption-inhibiting agents, and/or hormonal therapy) for a second period of time (either the same or different from the first period of time), and returned to alendronate therapy.

The dosage regime utilizing the claimed method is selected in accordance with a variety of factors including type, age, weight, sex, and medical condition of the patient; the severity of the condition to be treated; the route of administration; the renal and hepatic function of the patient; and the particular compound or salt thereof employed. An ordinarily skilled physician or clinician can readily determine and prescribe the effective amount of the drug required to prevent bone fractures.

Oral dosages of the present invention, when used to prevent bone fractures, will range from between 0.05 mg per kg of body weight per day (mg/kg/day) to about 1.0 mg/kg/day. Preferred oral dosages in humans may range from daily total dosages of about 2.5-50 mg/day over the effective treatment period, and a preferred amount is 2.5, 5, 10 or 20 mg/day. The dosages may be varied over a period of time, such that a patient may receive a high dose, such as 20 mg/day for a treatment period, such as two years, followed by a lower dose thereafter, such as 5 mg/day thereafter. Alternatively, a low dose (i.e., approximately 5 mg) may also be administered for a longer term with similar beneficial effects.

Alendronate may be administered in a single daily dose or in a divided dose. It is desirable for the dosage to be given in the absence of food, preferably from about 30 minutes to 2 hours prior to a meal, such as breakfast to permit adequate absorption.

In the methods of the present invention, the active ingredient is typically administered in admixture with suitable pharmaceutical diluents, excipients or carriers (collectively referred to herein as “carrier materials”) suitably selected with respect to the intended form of administration, i.e., oral tablets, capsules, elixirs, syrups and the like and consistent with conventional pharmaceutical practices. For example, for oral administration in the form of a tablet or capsule, the active ingredient can be combined with an oral, non-toxic, pharmaceutically acceptable inert carrier such as lactose, starch, sucrose, glucose, methyl cellulose, magnesium stearate, mannitol, sorbitol, and the like; for oral administration in liquid fcrm, the oral drug components can be combined with any oral, non-toxic, pharmaceutically acceptable inert carrier such as ethanol, glycerol, water and the like. Moreover, when desired or necessary, suitable binders, lubricants, disintegrating agents and coloring agents can also be incorporated into the mixture of active ingredient(s) and inert carrier materials. Suitable binders may include starch, gelatin, natural sugars such as glucose, anhydrous lactose, free-flow lactose, beta-lactose, and corn sweeteners, natural and synthetic gums, such as acacia, tragacanth or sodium alginate, carboxymethyl cellulose, polyethylene glycol, waxes, cros carmallose sodium and the like. Lubricants used in these dosage forms include sodium oleate, sodium stearate, magnesium stearate, sodium benzoate, sodium acetate, sodium chloride and the like. A particularly preferred tablet formulation is that described in U.S. Pat. No. 5,358,941 which is hereby incorporated by reference.

The compounds used in the instant method may also be coupled with soluble polymers as targetable drug carriers. Such polymers can include polyvinylpyrrolidone, pyran co-polymer, polyhydroxylpropyl-methacrylamide and the like.

The studies which were conducted in accordance with this invention selected patients based on their decreased spine BMD as compared to the overall population, and not a history of a prevalent vertebral fracture. This was done in order to more closely mirror the general osteoporotic population. Thus these patients were at a lower risk of incident vertebral fracture than patients typically recruited into fracture endpoint trials.

Various clinical endpoints were assessed in the course of this invention, such as:

STATURE—Height loss is a recognized clinical consequence of vertebral fractures. As a result of vertebral fractures due to osteoporosis, a patient may lose 10-20 cm over several years. Height loss results from vertebral collapse and kyphosis, which leads to reduced mobility and compression of the abdominal and thoracic cavities. Measurement of stature is a simple, inexpensive, easily repeated, radiation-free, and highly repeatable procedure. Importantly, stature is a continuous rather than a categorical variable, providing more power to detect differences between treatment groups. Although some individual patient variations in height may reflect changes in posture or in intervertebral disk spaces unrelated to osteoporosis, comparison of mean changes within treatment groups in a placebo-controlled, randomized, blinded study provides an accurate assessment of the effect of alendronate on vertebral fractures. Alendronate was found to significantly redue the observed mean decline in stature compared with placebo (p=0.005). Nonparametric and individual slope analyses were also significant (p=0.003 and p<0.001, respectively). All analytical approaches indicate that the rate of stature loss is reduced with alendronate treatment, and to a greater extent after three (as opposed to two) years of therapy.

Further, the mean decreases seen in placebo-treated patients with an incident vertebral fracture were substantially greater than those in similar patients on alendronaite. Patients who sustained at least one vertebral fracture lost a mean of 23.3 mm in stature in the placebo group, versus 5.9 mm in the alendronate group over three years. This marked difference implies that alendronate decreases not only the number of patients with incident fractures. but also decreases the average number of fractures and the average fracture severity. Thus, a further aspect of this invention is a method of decreasing the severity of fractures in patients who sustain a fracture by administering alendronate for a substantial period of time prior to the fracture.

VERTEBRAL FRACTURES—Calculations of prevalent and incident categorical vertebral fractures were performed by comparing each patient's baseline vertebral heights with a reference population (prevalent fracture) and with her follow-up heigiits (incident fractures). Only data from the true baselines were used to determine prevalent fractures. Any vertebral height ratio more than three standard deviations below its corresponding population reference value was defined as a prevalent vertebral fracture. An incident fracture was defined as greater than or equal to a 20% reduction from baseline vertebral height, with an absolute decrease of at least 4 mm in any vertebral height between baseline and follow-up. After three years of treatment, the observed reduction in vertebral fractures is both statistically significant (p=0.034) and clinically meaningful [48%; 95% C.I.=(72%, 5%)]. Reduction in vertebral fracture was consistent across multiple subgroup analysis, including by study, dose, age, (< or >65 years) and stratification by presence or absence of a prevalent vertebral fracture.

SPINAL DEFORMITY—The Spine Deformity Index (SDI) was calculated for each patient as described in Minne, et at., 1988, Bone and Min., 3:335-349, which is hereby incorporated by reference. Each individual vertebral height is divided by the corresponding height of the patient's fourth thoracic vertebra (T4) height (anterior, middle, or posterior) in order to generate a maximum of 39 vertebral height ratios. T4 was selected as the reference height because it is rarely fractured and can serve to adjust for differences in patient's height, as well as for differences in film focal distances between baseline and follow-up (which could artificially alter the apparent sizes of vertebral bodies between time points). Each of the height ratios is then compared with population norms, and for those ratios that fall below the minimum population norm, the absolute distances below the norm are summed to express the total SDI.

When SDI was utilized as a continuous measure of vertebral deformity, 41% of placebo patients showed progression in deformity, versus 33% of patients on alendronate (p=0.028). Additionally, there was a borderline significant difference (p=0.054) in the distribution of SDI changes between the two groups.

Also surprisingly, in accordance with this invention it was shown that the effect of reducing the risk of vertebral fracture is the same for elderly (at least 65 years of age) and non-elderly (age less than 65 years) patients. Thus another aspect of this invention is a method of decreasing the risk of vertebral fracture in elderly osteoporotic women by administering an effective amount of alendronate for a substantial period of time.

Further, it has been shown that the decrease of the risk of vertebral fractures due to alendronate treatment increases with time.

The following non-limiting examples are presented to better illustrate the invention.

### EXAMPLE 1

Postmenopausal women having a “low” lumbar spinal bone mineral density, defined as either a bone mineral density (BMD) of less than or equal to 0.92 g/cm² (+ or −0.02 g/cm²) as measured by Lunar DPX method, or less than or equal to 0.80 g/cm² (+ or −0.02 g/cm²) as measured by the Hologic QDR method are considered to have osteoporosis. This definition corresponds to a BMD of approximately two and one-half standard deviations below the mean BMD of mature pre-menopausal Caucasian women in the United States. Patients are otherwise in good health based on medical history, a physical examination and a laboratory screening evaluation. Only 20% of the enrolled women had vertebral fractures on entry.

Data was collected on a total of 881 patients from two study groups (cohorts), following virtually identical protocol design and procedures, except that one study was conducted in the United States, and the other was conducted in Canada, Mexico, Europe, Israel, South America, Australia and New Zealand. Data from the two groups was then pooled. 526 patients were treated with alendronate, from one of the following oral dosage regimes: A) 10 mg daily for three years; B) 5 mg for three years; or C) 20 mg for two years, followed by 5 mg for one year. 355 patients received placebo. Additionally, all patients received dietary evaluation and instruction on calcium intake. Almost all received calcium supplements to provide 500 mg elemental calcium (as carbonate) to ensure nutritional adequacy.

Assessment of vertebral fracture and vertebral deformity (SDI) was based on measurements from lateral spine x-rays, blinded to sequence. Lateral spine x-rays were taken at baseline, one, two and three years. The process of reading the x-rays involved a computerized entry of measurements taken at each of the vertebrae noted on the x-rays, a process known as digitization. Six landmarks on the bony process of each vertebra were noted, three along the superior edge and three along the inferior edge of each of 14 vertebrae, from the fourth thoracic vertebra to the fifth lumbar vertebra. A computer mouse with cross-hairs is used to enter the data as X,Y coordinates into a commercially available digitization board and computer software program, which computes the distance between landmarks (vertebral heights) in millimeters.

### EXAMPLE 2

Categorical Vertebral Fracture

Thirty nine women from Example 1 had at least one new vertebral fracture during the three years of study, as determined from their vertebral heights. Twenty-two of 355 (6.20%) women in the placebo group had a new vertebral fracture compared with 17 of 526 (3.23%) women in the alendronate group. This is a significantly lower amount (p=0.034)in the alendronate group.

The relative risk of incident fracture in the alendronate-treated versus the placebo treated patients was 0.52 (95% C.I.=[0.28, 0.95]). Additionally the magnitude of the fracture reduction after three years is greater than that seen after two years of treatment.

Moreover, among patients who experienced at least one incident vertebral fracture, the proportion of patients experiencing two or more fractures was far higher among placebo-treated patients (15/22; 68%) than those on alendronate (3/17; 18%). Because of the combination of fewer affected patients and fewer fractures per patient, the number of vertebral fractures per 100 patients was substantially lower in alendronate treated patients (4.2) than those on placebo (11.3).

Further, the group of alendronate-treated women who sustained an incident fracture had a less severe fractures than the group of placebo-treated women. TABLE 1, below shows the number of mild fractures (classified as end-plate deformity fractures) and severe fractures (crush or wedge fractures) in each group.

TABLE 1
Types of Fractures Sustained

| | Placebo | Alendronate |
|---|---|---|
| Mild fractures | 3/22 (13.6%) | 6/17 (35.3%) |
| Severe fractures | 19/22 (86.4%) | 11/17 (64.7%) |

### EXAMPLE 3

Spine Deformity Index

Results for changes in the Spine Deformity Index (SDI) were calculated as described in the specification. After three years, 41% of the women in the placebo group had an increase in vertebral deformity, compared to 33% of those in the alendronate group (p=0.028 by Chi-square test). This difference after three years is greater than that observed after two years.

Overall, the mean change from baseline was 0.082 and 0.041 for the placebo and the alendronate groups, respectively. In addition, for women with increased deformity, the mean changes were 0.212 and 0.143 for placebo and alendronate, respectively. The Wilcoxian rank sum test resulted in a borderline significant (p=0.054) difference in the distribution of SDI change from baseline between placebo and alendronate.

### EXAMPLE 4

### Stature

Height was measured in all patients using a Harpenden stadiometer, which precisely measures height to the nearest mm and is the most accurate method available to date. Height measurements were taken three times; if any two varied by more than 4 mm, a fourth and fifth measurement was taken. The average of the three (or five) measurements was used as the height value.

The mean change in stature after three years of treatment was −4.61 mm for the placebo group and −3.01 mm for the alendronate-treated group, which is a significant difference (p=0.005, 95% C.I.=[0.49, 2.71 mm]). The difference after three years was greater than the effect seen after only two years.

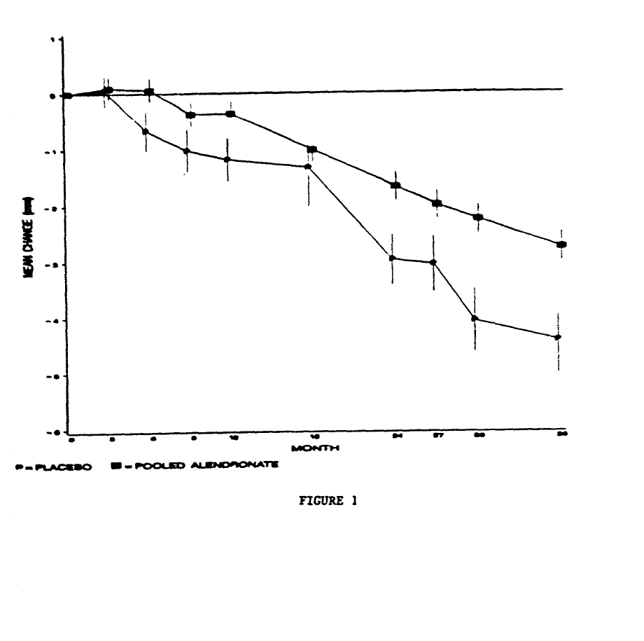

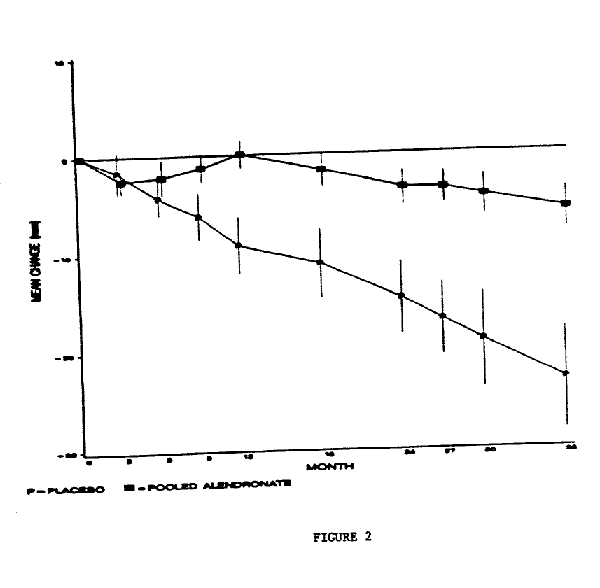

Further, a straight line was fitted to each individual's time-response profile to obtain an estimate of the slope for each individual. This is illustrated in FIGS. 1 and 2.

What is claimed is:

1. A method of reducing the risk of vertebral fractures in an osteoporotic female comprising administering an effective amount of alendronate or a pharmaceutically acceptable salt for a substantial period of time.

2. A method according to claim 1 wherein the alendronate is administered orally.

3. A method according to claim 2 wherein the dose is from 2.5 mg to 20 mg daily.

4. The method according to claim 3 wherein the alendronate is administered substantially daily for a period of at least two years.

5. The method according to claim 3 wherein the alendronate is administered substantially daily for a period of at least three years.

6. The method according to claim 1 wherein the female is elderly.

7. A method of reducing the severity of a fracture in patients who sustain a fracture by administering an effective amount of alendronate for a substantial period of time prior to the fracture.

8. A method according to claim 7 wherein the alendronate is administered orally.

9. A method according to claim 8 wherein the dose is from 2.5 mg to 20 mg daily.

10. The method according to claim 9 wherein the alendronate is administered substantially daily for a period of at least two years.

11. The method according to claim 9 wherein the alendronate is administered substantially daily for a period of at least three years.

12. The method according to claim 7 wherein the female is elderly.

13. A method of decreasing spinal deformity in osteoporotic women comprising administering an effective amount of alendronate for a substantial period of time.

14. A method according to claim 13 wherein the alendronate is administered orally.

15. A method according to claim 13 wherein the dose is from 2.5 mg to 20 mg daily.

16. The method according to claim 15 wherein the alendronate is administered substantially daily for a period of at least two years.

17. The method according to claim 15 wherein the alendronate is administered substantially daily for a period of at least three years.

18. The method according to claim 13 wherein the female is elderly.

19. A method of preventing loss of height in osteoporotic women by administering an effective amount of alendronate for a substantial period of time.

20. A method according to claim 19 wherein the alendronate is administered orally.

21. A method according to claim 20 wherein the dose is from 2.5 mg to 20 mg daily.

22. The method according to claim 21 wherein the alendronate is administered substantially daily for a period of at least two years.

23. The method according to claim 21 wherein the alendronate is administered substantially daily for a period of at least three years.

24. The method according to claim 19 wherein the female is elderly.
